(19) United States
(12) Patent Application Publication
(10) Pub. No.: US 2024/0268766 A1
(43) Pub. Date: Aug. 15, 2024

(54) BLOOD-PRESSURE-MEASURING DEVICE AND BLOOD-PRESSURE-MEASURING SYSTEM

(71) Applicant: OMRON HEALTHCARE Co., Ltd., Kyoto (JP)

(72) Inventors: Yasuhiro KAWABATA, Kyoto (JP); Kenji FUJII, Kyoto (JP); Naomi MATSUMURA, Kyoto (JP); Akito ITO, Kyoto (JP); Yuki SAKAGUCHI, Kyoto (JP); Takahide TANAKA, Kyoto (JP)

(21) Appl. No.: 18/442,775

(22) Filed: Feb. 15, 2024

Related U.S. Application Data
(63) Continuation of application No. PCT/JP2022/044935, filed on Dec. 6, 2022.

(30) Foreign Application Priority Data
Dec. 9, 2021 (JP) 2021-199787

(51) Int. Cl. A61B 5/00; A61B 5/0205; A61B 5/021; A61B 5/022; A61B 5/0245; A61B 5/282

(52) U.S. Cl. CPC A61B 5/7246; A61B 5/0205; A61B 5/282; A61B 5/6824; A61B 5/6831; A61B 5/742; A61B 5/02125; A61B 5/02141; A61B 5/02225; A61B 5/0245; A61B 2560/0228; A61B 2560/0238; A61B 2560/0468; A61B 2562/0247; A61B 2562/043

(57) ABSTRACT

A blood-pressure-measuring device includes: a feature amount acquisition unit that acquires one or more feature amounts related to estimation of a blood pressure value of a human body; a blood pressure value calculation unit that calculates an estimated blood pressure value based on the feature amount; an actually measured blood pressure value acquisition unit that acquires an actually measured blood pressure value measured by a method different from the calculation by the blood pressure value calculation unit; a calibration determination unit that determines whether or not the feature amount acquired by the feature amount acquisition unit deviates from a predetermined reference value, the calibration determination unit determining to acquire the actually measured blood pressure value when the calibration determination unit has determined that the feature amount deviates; and a calibration processing unit that estimates blood pressure value.

### CROSS-REFERENCE TO RELATED APPLICATIONS

This application is the U.S. national stage application filed pursuant to 35 U.S.C. 365(c) and 120 as a continuation of International Patent Application No. PCT/JP2022/044935, filed Dec. 6, 2022, which application claims priority to Japanese Patent Application No. 2021-199787, filed Dec. 9, 2021, which applications are incorporated herein by reference in their entireties.

### TECHNICAL FIELD

The present invention relates to a blood-pressure-measuring device and a blood-pressure-measuring system.

### BACKGROUND ART

Conventionally, as a method of measuring a blood pressure of a human body, there is known a technology of calculating a blood pressure estimation value based on a feature amount that can be acquired non-invasively and measuring the blood pressure using the estimation value. Specifically, for example, it is known that there is a correlation relationship between a pulse transit time (PTT), which is a time required for a pulse wave to propagate between two points on an artery, and a blood pressure. A device that non-invasively performs continuous blood pressure measurement based on the correlation relationship is proposed (for example, Patent Document 1).

Patent Document 1 discloses a blood-pressure-measuring device that measures a blood pressure by providing electrodes as an electro cardio graphic (ECG) sensors and a pulse wave sensor, such as a photo plethysmo graphic (PPG) sensor, on a belt unit wound around a target measurement site of a user and calculating a PTT based on a time difference between a waveform feature point in an electrocardiogram and a waveform feature point in a pulse wave signal. In this manner, with the configuration in which both the electrodes and the pulse wave sensor are provided on the belt unit, it is possible to mount the electrodes and the pulse wave sensor on the user by winding the belt unit around the user. For this reason, according to the technology described in Patent Document 1, it is possible to provide a blood-pressure-measuring device that can be easily attached to a user and can greatly reduce a load on the user when performing non-invasive continuous blood pressure measurement on a daily basis.

### CITATION LIST

### Patent Literature

Patent Document 1: JP 2019-154864 A

### SUMMARY OF INVENTION

### Technical Problem

Incidentally, when the blood pressure measurement (estimation) is performed based on the correlation relationship with the feature amount that can be acquired non-invasively as in the technology described in Patent Document 1, since the correlation relationship differs for each user or for each situation of the blood pressure measurement, it is required to measure an accurate blood pressure value at an appropriate timing and frequency and calibrate an algorithm for blood pressure estimation based on the blood pressure value. Patent Document 1 also describes that it is determined whether or not a recommended condition for the measurement of a blood pressure of a user for calibration is satisfied, and when the condition is satisfied, information for instructing the blood pressure measurement is output.

When the accurate blood pressure value is acquired for calibration as described above, an oscillometric method or the Korotkoff method is considered. However, compression of a target measurement site by a cuff is uncomfortable for a general user. Therefore, in the case where the blood pressure measurement is continuously performed at all times, when such blood pressure measurement for calibration is frequently performed, it brings a burden on the user. On the other hand, in a case where the blood pressure measurement (estimation) based on the feature amount is performed, to calculate a reliable and highly accurate measurement value (estimation value), it is necessary to appropriately perform calibration in accordance with various use states, such as an exercise state, an environment temperature, and a nutrition state of a user at the time of continuous measurement. That is, when the blood pressure calculation algorithm is calibrated in accordance with the user, it is desirable that the calibration is performed at a frequency necessary for accurate blood pressure value estimation and also at a frequency that minimizes the user's burden.

However, in an actual use environment, it is difficult for a user to intentionally try to change various measurement conditions (exercise intensity, environment temperature, nutrition state, and the like) and actually measure a blood pressure under various conditions, and perform calibration. In addition, when the calibration of the algorithm of the blood pressure calculation is uniformly requested with a threshold of a predetermined feature amount set in advance as in the related art, the frequency for performing the calibration does not change even though the accuracy of the blood pressure estimation is low (or conversely, sufficient accuracy is obtained). For this reason, it is not possible to achieve that the number of times of calibration is reduced so as not to perform unnecessary blood pressure measurement in accordance with individual characteristics of the user, measurement conditions, and the like or the number of times of calibration is increased so as to calculate an estimated blood pressure value with further high accuracy. That is, there was a problem that calibration cannot be performed at an appropriate frequency.

In view of the above-described circumstances, an object of the present invention is to provide a technology that allows optimizing a frequency of calibrating a blood pressure value calculation algorithm according to a user in a case where a blood pressure of a human body is estimated using a feature amount related to estimation of a blood pressure value.

### Solution to Problem

A blood-pressure-measuring device includes a feature amount acquisition unit, a blood pressure value calculation unit, an actually measured blood pressure value acquisition unit, a calibration determination unit, and a calibration processing unit. The feature amount acquisition unit acquires one or more feature amounts related to estimation of a blood pressure value of a human body. The blood pressure value calculation unit calculates an estimated blood pressure value based on the feature amount. The actually measured blood pressure value acquisition unit acquires an actually measured blood pressure value measured by a method different from the calculation by the blood pressure value calculation unit. The calibration determination unit determines whether or not the feature amount acquired by the feature amount acquisition unit deviates from a predetermined reference value. The calibration determination unit determines to acquire the actually measured blood pressure value when the calibration determination unit has determined that the feature amount deviates. The calibration processing unit calibrates a calculation algorithm of the estimated blood pressure value by the blood pressure value calculation unit using the actually measured blood pressure value. The calibration processing unit changes the reference value based on the actually measured blood pressure value acquired by the determination by the calibration determination unit and the estimated blood pressure value calculated using the feature amount deviated from the reference value.

Here, the feature amount includes, but is not limited to, waveform related data, such as a height at an inflection point, a gradient between inflection points, and an area of a predetermined portion in a waveform obtained from each of an electrocardiogram (ECG) and a pulse waveform, the feature amount calculated based on a plurality of pieces of waveform data, such as a PTT and a pulse arrival time (PAT), and besides biological information, such as data related to a heart rate. For example, information on attributes of individual patients, such as a height, an age, a weight, and medication history, and environment information, such as season and a temperature, are also included. “Calculating the estimated blood pressure value based on the feature amount” does not only mean calculating one estimated value from one specific feature amount but also includes calculating the estimated blood pressure value by combining a plurality of the feature amounts.

As described above, when the reference value for determining the necessity of calibration is changed based on the actually measured blood pressure value and the estimated blood pressure value, it is possible to improve accuracy of the blood pressure estimation by repeating the calibration of a blood pressure value calculation algorithm in accordance with a difference in individual characteristics of a user, and to optimize the frequency of the calibration of the blood pressure value calculation algorithm.

When a difference between the actually measured blood pressure value acquired by the determination by the calibration determination unit and the estimated blood pressure value calculated using the feature amount deviating from the reference value is a predetermined threshold or less, the calibration processing unit may change the reference value to a value that decreases a frequency determined when the actually measured blood pressure value is acquired. Alternatively, when a difference between the actually measured blood pressure value acquired by the determination by the calibration determination unit and the estimated blood pressure value calculated using the feature amount deviating from the reference value exceeds a predetermined threshold, the calibration processing unit may change the reference value to a value that increases a frequency determined when the actually measured blood pressure value is acquired.

When the blood pressure measurement for calibration is performed, the larger the difference between the estimated blood pressure value and the actually measured blood pressure value is, the more inappropriate the previous algorithm for blood pressure estimation is. Therefore, as described above, when the difference between the estimated blood pressure value and the actually measured blood pressure value is large, the reference value of the feature amount may be changed such that the frequency of the calibration increases (for example, when the value is set as the upper limit threshold, the value is decreased). On the other hand, when the difference between the estimated blood pressure value and the actually measured blood pressure value is small and the accuracy of blood pressure estimation is sufficient, the frequency of the calibration may be changed to decrease (for example, when the value is set as the upper limit threshold, the value is increased) to reduce the burden on the user. Thus, it is possible to easily optimize the number of times of the calibration processing without performing complicated processing.

The blood-pressure-measuring device may further include output means. When the calibration determination unit has determined to acquire the actually measured blood pressure value, information indicating that the actually measured blood pressure value is to be acquired may be output from the output means. The output means here can be, for example, a liquid crystal display, but may be another display means, such as an LED light, or output means other than the display means, such as a speaker or a vibration mechanism. With such a configuration, the user can easily recognize that the actually measured blood pressure value needs to be acquired.

The blood-pressure-measuring device may further include blood-pressure-measuring means that measures the actually measured blood pressure value. When the calibration determination unit has determined to acquire the actually measured blood pressure value, the actually measured blood pressure value acquisition unit acquires the actually measured blood pressure value by measuring the actually measured blood pressure value by the blood-pressure-measuring means. In this way, by further providing the blood-pressure-measuring means, it becomes possible to easily acquire the actually measured blood pressure value by measuring the blood pressure value when it becomes necessary to acquire the actually measured blood pressure value. As a result, it is possible to reduce the burden of blood pressure measurement using another device for actual measurement and the burden of data input.

The blood-pressure-measuring device may further include blood-pressure-measuring means for measuring the actually measured blood pressure value and operation input means. The actually measured blood pressure value acquisition unit acquires the actually measured blood pressure value by measuring the actually measured blood pressure value by the blood-pressure-measuring means when an input instructing the measurement of the actually measured blood pressure value is received via the operation input means.

According to this, since the blood pressure value is actually measured by the blood-pressure-measuring means by the operation of the user, the user can perform the blood pressure measurement after sufficiently arranging preparation for the measurement of the actually measured blood pressure value. That is, it is possible to prevent the measurement of the actually measured blood pressure value from being performed at a timing unassumed by the user or at an inconvenient timing.

The present invention can also be understood as a blood-pressure-measuring system having the following configuration. That is, a blood-pressure-measuring system includes feature amount acquisition means, blood pressure value calculation means, actually measured blood pressure value acquisition means, calibration determination means, and calibration processing means. The feature amount acquisition means acquires one or more feature amounts related to estimation of a blood pressure value of a human body. The blood pressure value calculation means calculates an estimated blood pressure value based on the feature amount. The actually measured blood pressure value acquisition means acquires an actually measured blood pressure value measured by a method different from the calculation by the blood pressure value calculation means. The calibration determination means determines whether or not the feature amount acquired by the feature amount acquisition means deviates from a predetermined reference value. The calibration determination means determines to acquire the actually measured blood pressure value when the calibration determination means has determined that the feature amount deviates. The calibration processing means calibrates a calculation algorithm of the estimated blood pressure value by the blood pressure value calculation means using the actually measured blood pressure value. The calibration processing means changes the reference value based on the actually measured blood pressure value acquired by the determination by the calibration determination means and the estimated blood pressure value calculated using the feature amount deviated from the reference value.

With such a configuration, it is possible to provide a function for solving the problem as the entire system without integrally configuring the respective means. For this reason, it is possible to reduce a burden on the user by a flexible method, such as narrowing down functions of devices owned and used by the user.

The blood-pressure-measuring system may include a measurement instrument that includes one or more sensors that detect at least the feature amount and an information processing device that includes at least the calibration processing means.

With such a configuration, configuration processing means that performs complicated arithmetic processing can be a separate terminal dedicated to information processing, and it is also possible to construct a cloud system that allows calibrating an algorithm of the measurement instrument of individual user by communicating with the measurement instrument used by the user and a server or the like installed in a remote place.

The measurement instrument may further include blood-pressure-measuring means for measuring the actually measured blood pressure value.

The measurement instrument may be a wearable device constantly attachable to a human body. The present invention is suitable for non-invasive continuous blood pressure measurement on a daily basis using the system having such a configuration.

Also, the configurations and processing described above can be combined with one another to constitute the present invention unless the combination leads to contradiction.

### Advantageous Effects of Invention

The present invention allows providing a technology that allows optimizing the frequency of calibrating the blood pressure value calculation algorithm according to the user in a case where the blood pressure of the human body is estimated using the feature amount related to the estimation of the blood pressure value.

### BRIEF DESCRIPTION OF DRAWINGS

FIG. 1 is a schematic view illustrating a blood-pressure-measuring device according to a first embodiment of the present invention.

FIG. 2 is a first diagram exemplifying an appearance of the blood-pressure-measuring device according to the first embodiment.

FIG. 3 is a second diagram exemplifying the appearance of the blood-pressure-measuring device according to the first embodiment.

FIG. 4 is a diagram exemplifying a cross-section of the blood-pressure-measuring device according to the first embodiment.

FIG. 5 is a block diagram exemplifying a hardware configuration of a control system of the blood-pressure-measuring device according to the first embodiment.

FIG. 6 is a block diagram exemplifying a software configuration of the blood-pressure-measuring device according to the first embodiment.

FIG. 7 is a flowchart depicting an example of a flow of processing by the blood-pressure-measuring device according to the first embodiment.

FIG. 8 is a schematic view illustrating a blood-pressure-measuring system according to a second embodiment of the present invention.

FIG. 9 is a block diagram schematically illustrating a functional configuration of each element of the blood-pressure-measuring system according to the second embodiment of the present invention.

FIG. 10 is a schematic view illustrating a blood-pressure-measuring system according to a third embodiment of the present invention.

FIG. 11 is a block diagram schematically illustrating a functional configuration of each element of the blood-pressure-measuring system according to the third embodiment.

### DESCRIPTION OF EMBODIMENTS

### First Embodiment

Embodiments of the present invention will be specifically described below with reference to the drawings. However, it should be noted that the dimensions, quality of materials, shape, relative arrangement, and the like of the components described in the embodiments below are not intended to limit the scope of this invention to them alone, unless otherwise stated.

### Overview

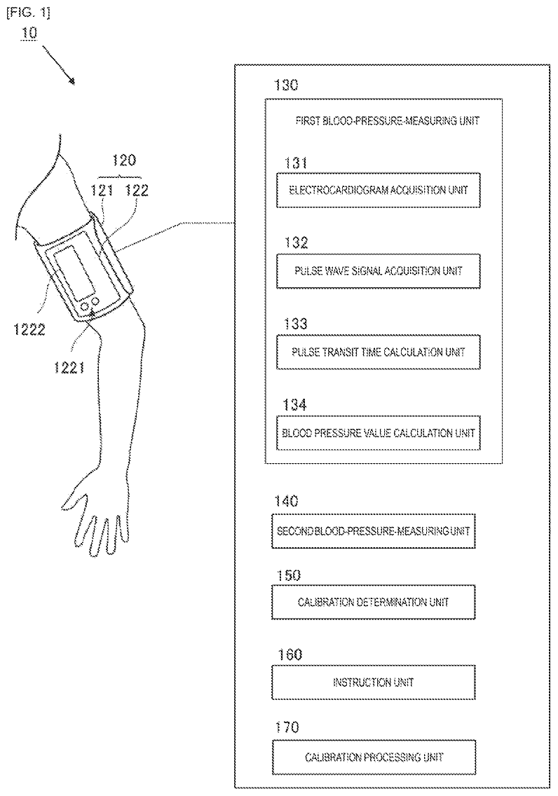

FIG. 1 is a schematic view exemplifying a blood-pressure-measuring device 10 according to one embodiment. The blood-pressure-measuring device 10 is a wearable device and is attached to an upper arm as a target measurement site of a user. As an outline, the blood-pressure-measuring device 10 includes a belt unit 120, a first blood-pressure-measuring unit 130, a second blood-pressure-measuring unit 140, a calibration determination unit 150, an instruction unit 160, and a calibration processing unit 170.

The belt unit 120 includes a belt 121 and a body 122. The belt 121 is a band-like member that is attached around an upper arm and is sometimes referred to by another name, such as a band or a cuff. The belt 121 has an inner circumferential surface and an outer circumferential surface. The inner circumferential surface is a surface that comes into contact with the upper arm of the user in a state in which the user attaches the blood-pressure-measuring device 10 (hereinafter, simply referred to as an “attachment state”), and the outer circumferential surface is a surface on a side opposite to the inner circumferential surface.

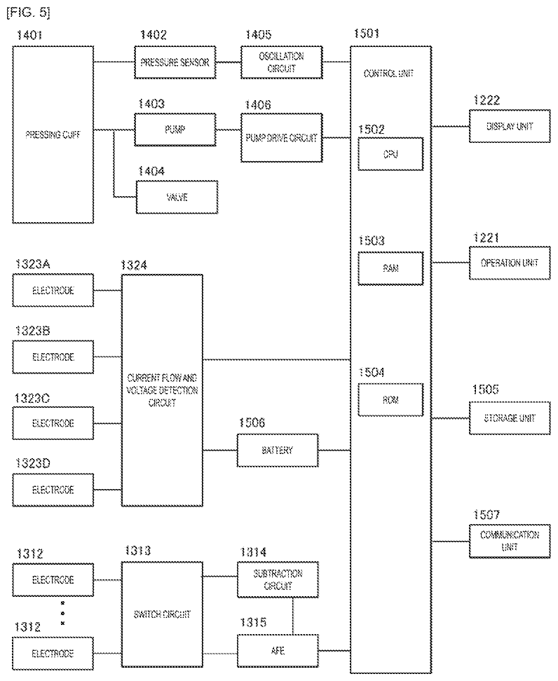

The body 122 is mounted on the belt 121. The body 122 accommodates components, such as a control unit 1501 (illustrated in FIG. 5) described below, together with an operation unit 1221 and a display unit 1222. The operation unit 1221 is an input device that allows the user to input an instruction to the blood-pressure-measuring device 10. In the example of FIG. 1, the operation unit 1221 includes a plurality of push buttons. The display unit 1222 is a display device displaying information, such as a message prompting blood pressure measurement and a blood pressure measurement result. As a display device, for example, a liquid crystal display (LCD), an organic light emitting diode (OLED) display, and the like can be used. A touch screen that also serves as a display device and an input device may be used. The body 122 may be provided with a sound emitter, such as a speaker or a piezoelectric sounder. The body 122 may be provided with a microphone to allow the user to input instructions by sounds.

The first blood-pressure-measuring unit 130 non-invasively measures the pulse transit time of the user and calculates the blood pressure value based on the measured pulse transit time (PTT). Hereinafter, the blood pressure value calculated based on the pulse transit time in this way is also referred to as an estimated blood pressure value. The first blood-pressure-measuring unit 130 can perform continuous blood pressure measurement for obtaining the blood pressure value for each beat.

The second blood-pressure-measuring unit 140 performs blood pressure measurement using a method different from that of the first blood-pressure-measuring unit 130. Specifically, the second blood-pressure-measuring unit 140 performs the blood pressure measurement at a specific timing, for example, in response to an operation by the user by, for example, an oscillometric method or the Korotkoff method. The second blood-pressure-measuring unit 140 cannot perform the continuous blood pressure measurement, but can measure the blood pressure more accurately than the first blood-pressure-measuring unit 130. Hereinafter, the blood pressure value measured by the second blood-pressure-measuring unit 140 is also referred to as an actually measured blood pressure value.

The first blood-pressure-measuring unit 130 includes respective functional modules of an electrocardiogram acquisition unit 131, a pulse wave signal acquisition unit 132, a pulse transit time calculation unit 133, and a blood pressure value calculation unit 134.

The electrocardiogram acquisition unit 131 includes a plurality of electrodes and acquires an electrocardiogram (ECG) of the user using these electrodes. The electrocardiogram represents electrical activity of a heart. The electrodes are provided on the belt unit 120. For example, the electrodes are disposed on the inner circumferential surface of the belt 121 such that the electrodes are in contact with a skin of the upper arm of the user in the attachment state.

The pulse wave signal acquisition unit 132 includes a pulse wave sensor and acquires a pulse wave signal representing a pulse wave of the user using the pulse wave sensor. The pulse wave sensor is provided on the belt unit 120. For example, the pulse wave sensor is disposed on the inner circumferential surface of the belt 121 such that the pulse wave sensor is in contact with the skin of the upper arm of the user in the attachment state. Note that some types of pulse wave sensors, such as pulse wave sensors based on a radio wave method described later, do not need to be in contact with the skin of the upper arm of the user in the attachment state.

The pulse transit time calculation unit 133 calculates the pulse transit time based on a time difference between a waveform feature point in the electrocardiogram acquired by the electrocardiogram acquisition unit 131 and a waveform feature point in the pulse wave signal acquired by the pulse wave signal acquisition unit 132. For example, the pulse transit time calculation unit 133 calculates the time difference between the waveform feature point in the electrocardiogram and the waveform feature point in the pulse wave signal and outputs the calculated time difference as the pulse transit time. In the present embodiment, the pulse transit time corresponds to a time required for a pulse wave to propagate through an artery, from the heart to the upper arm (specifically, the position where the pulse wave sensor is disposed).

The blood pressure value calculation unit 134 calculates the blood pressure value based on the pulse transit time calculated by the pulse transit time calculation unit 133 and a blood pressure calculation formula. The blood pressure calculation formula is a relational formula that represents a correlation between the pulse transit time and the blood pressure. An example of a blood pressure calculation formula is illustrated below.

$$SBP = {A}_{1} / {PTT}^{2} + {A}_{2} … \qquad ( 1 )$$

Here, SBP represents systolic blood pressure, PTT represents the pulse transit time, and A₁ and A₂ are parameters.

The pulse transit time calculation unit 133 can calculate the pulse transit time for each beat, and thus the blood pressure value calculation unit 134 can calculate the blood pressure value for each beat.

The calibration determination unit 150 monitors a predetermined feature amount (for example, PTT in the present embodiment) acquired by the first blood-pressure-measuring unit 130, and determines whether or not the feature amount deviates from a predetermined reference value (for example, upper and lower limit thresholds). Then, when the feature amount is determined to deviate from the predetermined reference value, it is determined to acquire the actually measured blood pressure value of the user.

When the calibration determination unit 150 has determined to acquire the actually measured blood pressure value, the instruction unit 160 outputs information for instructing the blood pressure measurement by the second blood-pressure-measuring unit 140. For example, the instruction unit 160 outputs a notification sound (for example, a melody) through a sounder and causes the display unit 1222 to display a message “Please measure blood pressure.” When the user presses a predetermined button in response to the instruction from the instruction unit 160, the blood pressure measurement by the second blood-pressure-measuring unit 140 is performed. The blood pressure measurement by the second blood-pressure-measuring unit 140 will be described later.

The calibration processing unit 170 calibrates the blood pressure calculation formula (1) based on the actually measured blood pressure value measured by the second blood-pressure-measuring unit 140. Since the correlation relationship between the pulse transit time represented by the blood pressure calculation formula and the blood pressure differs for each individual user, it is necessary to calibrate the blood pressure calculation formula for the user. The blood pressure calculation formula is calibrated (specifically, the parameters A₁ and A₂ are determined) based on the actually measured blood pressure value obtained by the second blood-pressure-measuring unit 140. The calibration of the blood pressure calculation formula will be described in detail later.

As described above, in the blood-pressure-measuring device 10, the plurality of electrodes used to acquire the electrocardiogram and the pulse wave sensor used to acquire the pulse wave signal are both provided in the belt unit 120. This allows the electrodes and the pulse wave sensor to be mounted on the user simply by winding the belt unit 120 around the upper arm. Thus, the device can be easily attached to the user, and a feeling of rejection of the user for the attachment of the blood-pressure-measuring device 10 can be reduced.

Further, the time difference between the waveform feature point in the electrocardiogram and the waveform feature point in the pulse wave signal regarding the upper arm can be calculated as the pulse transit time. The pulse transit time obtained by the blood-pressure-measuring device 10 becomes a value larger than that when the pulse transit time between two points in the upper arm is measured. In other words, a further long pulse transit distance is ensured. Accordingly, an influence of an error generated in calculating the time difference between the waveform feature point in the electrocardiogram and the waveform feature point in the pulse wave signal on the pulse transit time is reduced and the pulse transit time can be accurately measured. As a result, reliability of the blood pressure value obtained by the blood pressure measurement based on the pulse transit time is improved.

### Configuration Example

Hereinafter, the blood-pressure-measuring device 10 will be described more specifically.

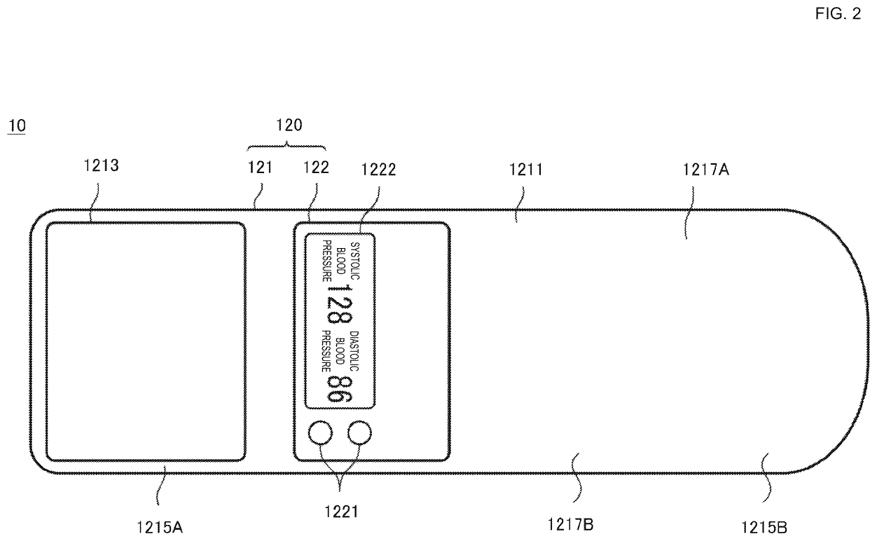

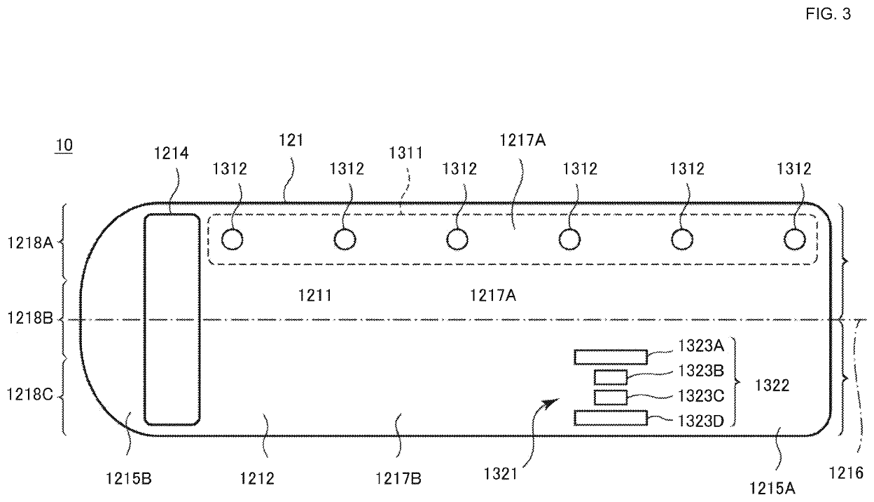

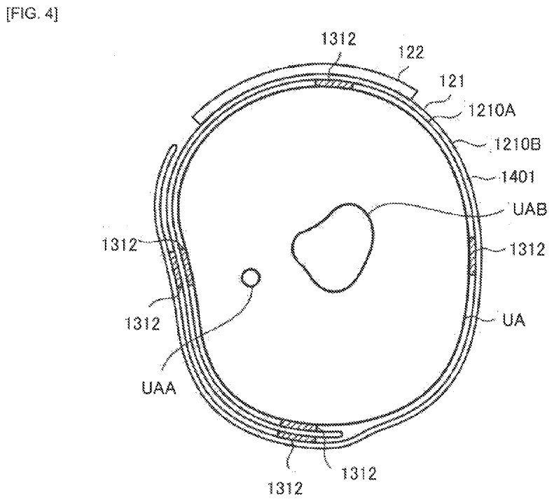

An example of the configuration of the blood-pressure-measuring device 10 according to the present embodiment will be described with reference to FIG. 2 to FIG. 6. FIG. 2 and FIG. 3 are plan views illustrating the appearance of the blood-pressure-measuring device 10. Specifically, FIG. 2 illustrates the blood-pressure-measuring device 10 viewed from an outer circumferential surface 1211 side of the belt 121 in an expanded state of the belt 121, and FIG. 3 illustrates the blood-pressure-measuring device 10 viewed from an inner circumferential surface 1212 side of the belt 121 in an expanded state of the belt 121. FIG. 4 illustrates a cross-section of the blood-pressure-measuring device 10 in the attachment state.

The belt 121 includes an attachment member allowing the belt 121 to be detachably attached to the upper arm. In the example illustrated in FIG. 2 and FIG. 3, the attachment member is a surface fastener including a loop surface 1213 including the large number of loops and a hook surface 1214 including a plurality of hooks. The loop surface 1213 is disposed on the outer circumferential surface 1211 of the belt 121 at an end portion 1215A in a longitudinal direction of the belt 121. The longitudinal direction corresponds to the circumferential direction of the upper arm in the attachment state. The hook surface 1214 is disposed on the inner circumferential surface 1212 of the belt 121 at an end portion 1215B in the longitudinal direction of the belt 121. The end portion 1215B is opposed to the end portion 1215A in the longitudinal direction of the belt 121. When the loop surface 1213 and the hook surface 1214 are pressed against one another, the loop surface 1213 and the hook surface 1214 are joined. In addition, pulling the loop surface 1213 and the hook surface 1214 away from one another separates the loop surface 1213 and the hook surface 1214.

As illustrated in FIG. 3, an electrode group 1311 for measuring the electrocardiogram is disposed on the inner circumferential surface 1212 of the belt 121. In the example of FIG. 3, the electrode group 1311 has six electrodes 1312 aligned at regular intervals in the longitudinal direction of the belt 121.

The interval between the electrodes 1312 is set, for example, to a quarter of the circumference of the upper arm of the user assumed to have the thinnest arm. In this arrangement, as illustrated in FIG. 4, for the user assumed to have the thinnest arm, four of the six electrodes 1312 contact an upper arm UA in the attachment state and are located at regular intervals on the circumference of the upper arm, and the remaining two electrodes 1312 contact the outer circumferential surface of the belt 121. In FIG. 4, a humerus UAB and a brachial artery UAA are illustrated. For a user assumed to have the thickest arm, all the six electrodes 1312 contact the upper arm UA in the attachment state.

Note that the number of electrodes 1312 is not limited to six, and may be two to five or seven or more. When the two or three electrodes 1312 are in contact with the upper arm, the electrocardiogram is not successfully measured depending on the attachment state in some cases. When the electrocardiogram is not successfully measured, for example, a message is displayed on the display unit 1222, and the blood-pressure-measuring device 10 needs to be re-attached to the user. To avoid situations in which the electrocardiogram cannot be measured, it is desired that at least the four electrodes 1312 contact the upper arm in the attachment state.

The closer the electrode 1312 is located to the heart in the attachment state, the greater a signal representing the electrical activity of the heart obtained using the electrode 1312 becomes, that is, a signal to noise ratio (SN ratio) becomes higher. Preferably, as illustrated in FIG. 3, the electrodes 1312 are disposed in a central side portion 1217A of the belt 121. The central side portion 1217A is a portion located closer to the central side (shoulder side) than a center line 1216 in the attachment state. More preferably, the electrode 1312 is disposed at a central side end portion 1218A of the belt 121. The central side end portion 1218A is an end portion located on the central side in the attachment state, and a width of the central side end portion 1218A is, for example, one-third of the full width of the belt 121.

A sensor unit 1322 of a pulse wave sensor 1321 for measuring the pulse wave is further disposed on the inner circumferential surface 1212 of the belt 121. In the example of FIG. 3, the sensor unit 1322 includes a pair of electrodes 1323A, 1323D for energizing the upper arm and a pair of electrodes 1323B, 1323C for detecting a voltage. The electrodes 1323A, 1323B, 1323C, 1323D are arranged in that order in the width direction of the belt 121. The width direction of the belt 121 corresponds to a direction along the brachial artery UAA in the attachment state.

The farther the sensor unit 1322 is located from the heart in the attachment state, the longer the pulse transit distance is and the greater the measurement value of the pulse transit time is. Therefore, the error generated in calculating the time difference between the waveform feature point in the electrocardiogram and the waveform feature point in the pulse wave signal is relatively smaller than the pulse transit time, and the pulse transit time can be accurately measured. Preferably, the sensor unit 1322 is disposed in a peripheral side portion 1217B of the belt 121. The peripheral side portion 1217B is a portion located closer to the peripheral side (elbow side) than the center line 1216 in the attachment state. More preferably, the sensor unit 1322 is disposed at a peripheral side end portion 1218C of the belt 121. The peripheral side end portion 1218C is an end portion located on the peripheral side in the attachment state, and a width of the peripheral side end portion 1218C is, for example, one-third of the full width of the belt 121. A portion between the central side end portion 1218A and the peripheral side end portion 1218C is referred to as an intermediate portion 1218B.

As illustrated in FIG. 4, the belt 121 includes an inner cloth 1210A, an outer cloth 1210B, and a pressing cuff 1401 provided between the inner cloth 1210A and the outer cloth 1210B. The pressing cuff 1401 is a band-like body that is long in the longitudinal direction of the belt 121 such that the pressing cuff 1401 can surround the upper arm. For example, the pressing cuff 1401 is configured as a fluid bag by placing two stretchable polyurethane sheets to be opposed in the thickness direction and welding edge portions of the polyurethane sheets. The electrode group 1311 and the sensor unit 1322 are provided in the inner cloth 1210A such that the electrode group 1311 and the sensor unit 1322 are located between the pressing cuff 1401 and the upper arm UA in the attachment state.

FIG. 5 illustrates an example of a hardware configuration of a control system of the blood-pressure-measuring device 10 according to the present embodiment. In the example of FIG. 5, in addition to the operation unit 1221 and the display unit 1222 described above, the body 122 includes the control unit 1501, a storage unit 1505, a battery 1506, a switch circuit 1313, a subtraction circuit 1314, an analog front end (AFE) 1315, a pressure sensor 1402, a pump 1403, a valve 1404, an oscillation circuit 1405, and a pump drive circuit 1406. In addition to the sensor unit 1322 described above, the pulse wave sensor 1321 includes an energization and voltage detection circuit 1324. In this example, the energization and voltage detection circuit 1324 is mounted on the belt 121.

The control unit 1501 includes a Central Processing Unit (CPU) 1502, a Random Access Memory (RAM) 1503, a Read Only Memory (ROM) 1504, and the like and controls each component according to information processing. The storage unit 1505 is, for example, an auxiliary storage device, such as a hard disk drive (HDD) or a semiconductor memory (for example, a flash memory) and non-volatilely stores programs executed by the control unit 1501 (including, for example, a pulse transit time measurement program and a blood pressure measurement program), settings data necessary for executing the programs, results of blood pressure measurement, and the like. A storage medium provided in the storage unit 1505 is a medium that accumulates information such as a recorded program by an electrical, magnetic, optical, mechanical, or chemical action such that a computer, other devices, a machine, or the like can read the information such as the recorded program. Note that a portion or all of the programs may be stored in the ROM 1504.

The battery 1506 supplies electric power to components, such as the control unit 1501. The battery 1506 is, for example, a rechargeable battery.

The electrodes 1312 included in the electrode group 1311 are each connected to an input terminal of the switch circuit 1313. Respective two output terminals of the switch circuit 1313 are connected to two input terminals of the subtraction circuit 1314. The switch circuit 1313 receives a switch signal from the control unit 1501 and connects the two electrodes 1312 designated by the switch signal to the subtraction circuit 1314. The subtraction circuit 1314 subtracts, from a potential input from one input terminal, a potential input from the other input terminal. The subtraction circuit 1314 outputs, to the AFE 1315, a potential difference signal that represents the potential difference between the two connected electrodes 1312. The subtraction circuit 1314 is, for example, an instrumentation amplifier. The AFE 1315 includes, for example, a low-pass filter (LPF), an amplifier, and an analog-to-digital converter. The potential difference signal is filtered by the LPF, amplified by the amplifier, and converted to a digital signal by the analog-to-digital converter. The potential difference signal converted to a digital signal is provided to the control unit 1501. The control unit 1501 acquires, from the AFE 1315, the potential difference signal output in a time-series manner as the electrocardiogram.

The energization and voltage detection circuit 1324 flows a high-frequency constant current between the electrodes 1323A, 1323D. In this example, the current has a frequency of 50 kHz and a current value of 1 mA. The energization and voltage detection circuit 1324 detects a voltage across the electrodes 1323B, 1323C and generates a detection signal, in a state in which a current flows between the electrodes 1323A, 1323D. The detection signal represents a change in electrical impedance due to a pulse wave that propagates through a portion of the artery that the electrodes 1323B, 1323C are opposed to. The energization and voltage detection circuit 1324 performs signal processing including rectifying, amplifying, filtering, and analog-to-digital conversion on the detection signal and supplies the detection signal to the control unit 1501. The control unit 1501 acquires, from the energization and voltage detection circuit 1324, the detection signal output in the time-series manner as the pulse wave signal.

The pressure sensor 1402 is connected to the pressing cuff 1401 via a pipe, and the pump 1403 and the valve 1404 are connected to the pressing cuff 1401 via a pipe. Note that these pipes may be one common pipe or may be separate pipes. The pump 1403 is, for example, a piezoelectric pump and feeds air as a fluid to the pressing cuff 1401 through the pipe to increase a pressure inside the pressing cuff 1401. The valve 1404 is mounted on the pump 1403, and opening and closing of the valve 1404 is controlled according to an operation state (on/off) of the pump 1403. Specifically, the valve 1404 is in a closed state when the pump 1403 is turned on, and the valve 1404 is in an open state when the pump 1403 is turned off. When the valve 1404 is in the open state, the pressing cuff 1401 is in communication with the atmosphere, and the air in the pressing cuff 1401 is discharged into the atmosphere. The valve 1404 has a function of a check valve, and air does not flow back through it. The pump drive circuit 1406 drives the pump 1403 based on a control signal received from the control unit 1501.

The pressure sensor 1402 detects the pressure in the pressing cuff 1401 (also referred to as a cuff pressure) and generates an electric signal representing the cuff pressure. The cuff pressure is, for example, pressure based on atmospheric pressure as a reference. The pressure sensor 1402 is, for example, a piezoresistive pressure sensor. The oscillation circuit 1405 oscillates based on the electrical signal from the pressure sensor 1402 and outputs, to the control unit 1501, a frequency signal having a frequency in accordance with the electrical signal. In this example, the output of the pressure sensor 1402 is used for controlling the pressure of the pressing cuff 1401 and for calculating a blood pressure value (including a systolic blood pressure and a diastolic blood pressure) using an oscillometric method.

The pressing cuff 1401 may be used for adjusting the contact state between the electrode 1312 or the sensor unit 1322 of the pulse wave sensor 1321 and the upper arm UA. For example, during execution of the blood pressure measurement based on the pulse transit time, the pressing cuff 1401 is maintained in a state in which some air is accommodated therein. As a result, the electrode 1312 and the sensor unit 1322 of the pulse wave sensor 1321 are reliably in contact with the upper arm UA.

In the example illustrated in FIG. 2 to FIG. 5, the electrode group 1311, the switch circuit 1313, the subtraction circuit 1314, and the AFE 1315 correspond to the electrocardiogram acquisition unit 131 of the first blood-pressure-measuring unit 130 illustrated in FIG. 1, and the pulse wave sensor 1321 (the electrodes 1323 and the energizing and voltage detection circuit 1324) corresponds to the pulse wave signal acquisition unit 132 of the first blood-pressure-measuring unit 130. Also, the pressing cuff 1401, the pressure sensor 1402, the pump 1403, the valve 1404, the oscillation circuit 1405, and the pump drive circuit 1406 correspond to the second blood-pressure-measuring unit 140.

Also, with respect to a specific hardware configuration of the blood-pressure-measuring device 10, components can be omitted, replaced, or added as appropriate in accordance with embodiments. For example, the control unit 1501 may include a plurality of processors. The blood-pressure-measuring device 10 may include a communication unit 1507 for communicating with an external device such as a portable terminal of the user (for example, a smartphone). The communication unit 1507 includes a wired communication module and/or a wireless communication module. As a wireless system, for example, Bluetooth (trade name), Bluetooth Low Energy (BLE), or the like can be adopted.

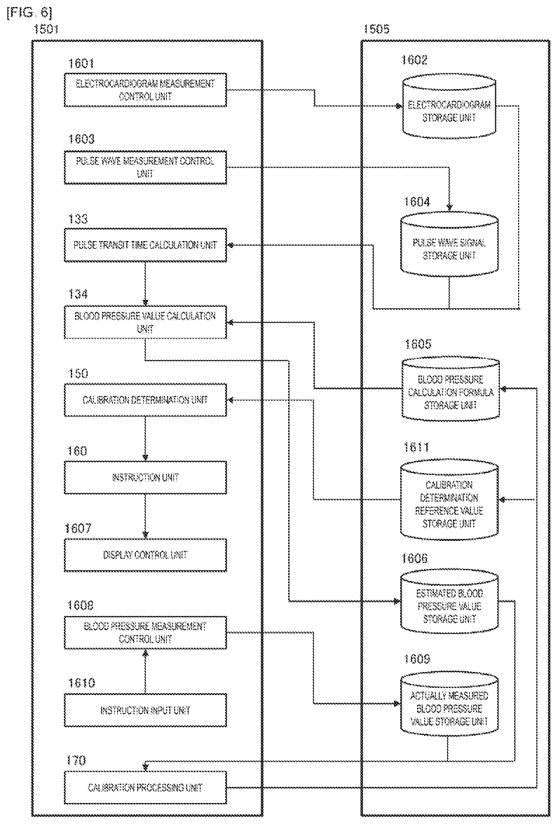

FIG. 6 exemplifies an example of a software configuration of the blood-pressure-measuring device 10 according to the present embodiment. In the example of FIG. 6, the blood-pressure-measuring device 10 includes an electrocardiogram measurement control unit 1601, an electrocardiogram storage unit 1602, a pulse wave measurement control unit 1603, a pulse wave signal storage unit 1604, the pulse transit time calculation unit 133, the blood pressure value calculation unit 134, a blood pressure calculation formula storage unit 1605, an estimated blood pressure value storage unit 1606, the calibration determination unit 150, the instruction unit 160, a blood-pressure-measuring control unit 1608, an actually measured blood pressure value storage unit 1609, a display control unit 1607, an instruction input unit 1610, the calibration processing unit 170, and a calibration determination reference value storage unit 1611. The electrocardiogram measurement control unit 1601, the pulse wave measurement control unit 1603, the pulse transit time calculation unit 133, the blood pressure value calculation unit 134, the calibration determination unit 150, the instruction unit 160, the blood-pressure-measuring control unit 1608, the display control unit 1607, the instruction input unit 1610, and the calibration processing unit 170 perform the following processing when the control unit 1501 of the blood-pressure-measuring device 10 executes programs stored in the storage unit 1505. When the control unit 1501 executes the program, the control unit 1501 unfolds the program in the RAM 1503. Then, the control unit 1501 causes the CPU 1502 to interpret and execute the program unfolded in the RAM 1503 to control each component. The electrocardiogram storage unit 1602, the pulse wave signal storage unit 1604, the blood pressure calculation formula storage unit 1605, the estimated blood pressure value storage unit 1606, the actually measured blood pressure value storage unit 1609, and the calibration determination reference value storage unit 1611 are achieved by the storage unit 1505.

The electrocardiogram measurement control unit 1601 controls the switch circuit 1313 to acquire the electrocardiogram. Specifically, the electrocardiogram measurement control unit 1601 generates a switch signal for selecting the two electrodes 1312 from the six electrodes 1312 and provides the switch signal to the switch circuit 1313. The electrocardiogram measurement control unit 1601 acquires the potential difference signal acquired using the two selected electrodes 1312 and stores the time-series data of the acquired potential difference signal in the electrocardiogram storage unit 1602 as the electrocardiogram.

When the user attaches the blood-pressure-measuring device 10 to the upper arm, the electrocardiogram measurement control unit 1601 determines an optimum electrode pair for acquiring the electrocardiogram. For example, the electrocardiogram measurement control unit 1601 acquires the electrocardiogram for each of all electrode pairs and determines an electrode pair that provides an electrocardiogram with the greatest amplitude of an R wave as the optimal electrode pair. Thereafter, the electrocardiogram measurement control unit 1601 measures the electrocardiogram using the optimal electrode pair.

The pulse wave measurement control unit 1603 controls the energization and voltage detection circuit 1324 to acquire the pulse wave signal. Specifically, the pulse wave measurement control unit 1603 instructs the energization and voltage detection circuit 1324 to flow a current between the electrodes 1323A, 1323D and acquires a detection signal indicating the voltage between the electrodes 1323B, 1323C detected with the current flowing between the electrodes 1323A, 1323D. The pulse wave measurement control unit 1603 stores the time-series data of the detection signal in the pulse wave signal storage unit 1604 as the pulse wave signal.

The pulse transit time calculation unit 133 reads the electrocardiogram from the electrocardiogram storage unit 1602, reads the pulse wave signal from the pulse wave signal storage unit 1604, and calculates the pulse transit time based on the time difference between the waveform feature point in the electrocardiogram and the waveform feature point in the pulse wave signal. For example, the pulse transit time calculation unit 133 detects the time (point in time) of a peak point corresponding to the R wave from the electrocardiogram, detects the time (point in time) of a rising point from the pulse wave signal, and subtracts the time of the peak point from the time of the rising point to calculate the difference as the pulse transit time.

Note that the pulse transit time calculation unit 133 may correct the above-described time difference based on a preejection period (PEP) and output the corrected time difference as the pulse transit time. For example, with the preejection period considered to be constant, the pulse transit time calculation unit 133 may calculate the pulse transit time by subtracting a predetermined value from the time difference described above.

The peak point corresponding to the R wave is an example of a waveform feature point in the electrocardiogram. The waveform feature point in the electrocardiogram may be a peak point corresponding to a Q wave or a peak point corresponding to an S wave. Since the R wave appears as a distinct peak compared to the Q or S wave, the time of the R wave peak point can be more accurately identified. Thus, preferably, the R wave peak point is used as the waveform feature point in the electrocardiogram. Additionally, the rising point is an example of a waveform feature point in the pulse wave signal. The waveform feature point in the pulse wave signal may be the peak point. Since the pulse wave signal gradually changes with time, an error is likely to occur when the time of the waveform feature point is identified in the pulse wave signal.

With reference to FIG. 6, the blood pressure value calculation unit 134 calculates the estimated blood pressure value based on the pulse transit time calculated by the pulse transit time calculation unit 133 and the blood pressure calculation formula. The blood pressure value calculation unit 134 uses, as the blood pressure calculation formula, an algorithm (specifically, for example, the above-described formula (1)) for calculating the blood pressure value stored in the blood pressure calculation formula storage unit 1605. The blood pressure value calculation unit 134 causes the estimated blood pressure value storage unit 1606 to store the calculated blood pressure value in association with time information.

Note that the blood pressure calculation formula is not limited to Formula (1) above. The blood pressure calculation formula may be, for example, the following formula.

$$SBP = {B}_{1} / {PTT}^{2} + {B}_{2} / PTT + {B}_{3} \times PTT + {B}_{4} … \qquad ( 2 )$$

Here, B₁, B₂, B₃, and B₄ are parameters.

The calibration determination unit 150 determines whether or not a recommended condition for measurement of the blood pressure of the user is satisfied based on a predetermined feature amount related to blood pressure estimation, for example, the pulse transit time calculated by the pulse transit time calculation unit 133 and a predetermined reference value for the feature amount stored in the calibration determination reference value storage unit 1611. Even when the blood pressure calculation formula is calibrated at the start of use of the device, the accuracy of the calculated estimated blood pressure value is considered to decreases in a situation where the feature amount related to the calculation of the estimated blood pressure value deviates from the predetermined reference value (upper and lower limit thresholds). Therefore, in such a case, it is desirable to perform accurate blood pressure measurement by the second blood-pressure-measuring unit 140, to check the accuracy of the estimated blood pressure value by comparing the actually measured blood pressure value with the estimated blood pressure value, and to calibrate the blood pressure calculation formula when the accuracy is low (that is, the difference between the actually measured blood pressure value and the estimated blood pressure value is large).

As another example, the calibration determination unit 150 may determine whether or not a blood pressure change rate exceeds a threshold as the predetermined feature amount. The blood pressure change rate is, for example, a change amount of a blood pressure value in a unit time. Specifically, the calibration determination unit 150 determines whether or not a difference obtained by subtracting the blood pressure value before the unit time from the latest blood pressure value exceeds a threshold. Assuming that the latest systolic blood pressure value is SBP₀, the systolic blood pressure value before the unit time is SBP₁, and a threshold is Vₜₕ, the calibration determination unit 150 determines whether or not a conditional expression SBP₀−SBP₁>Vₜₕ is satisfied. The unit time is, for example, 30 seconds, and the threshold is 20 [mmHg], for example. When the latest value of the pulse transit time is PTT₀ and the value of the pulse transit time before the unit time is PTT₁, transformation of the above conditional expression using Expression (1) to be A₁(1/PTT₀²−1/PTT₁²)>Vₜₕ.

That is, the calibration determination unit 150 may use the pulse transit time itself, or may use the blood pressure value calculated based on the pulse transit time. The calibration determination unit 150 may determine whether or not a difference obtained by subtracting the blood pressure value before the predetermined number of heart beats (for example, 30 beats before) from the latest blood pressure value exceeds a threshold. In another example, the calibration determination unit 150 determines whether or not the value of the latest systolic blood pressure exceeds a threshold (for example, 150 [mmHg]). The threshold may be fixed or may be variable. For example, the threshold is set to a higher value as an average blood pressure of the user is higher.

When the calibration determination unit 150 has determined to acquire the actually measured blood pressure value, the instruction unit 160 outputs information for instructing the blood pressure measurement by the second blood-pressure-measuring unit 140. For example, the instruction unit 160 gives an instruction signal to the display control unit 1607 so as to cause the display unit 1222 to display a message prompting the blood pressure measurement. Furthermore, the instruction unit 160 outputs a control signal for controlling a drive circuit that drives a sounder to generate a notification sound. The instruction unit 160 may transmit the instruction signal to the mobile terminal of the user via the communication unit 1507 to prompt the user to perform the blood pressure measurement via the mobile terminal.

The instruction input unit 1610 receives an instruction input from the user using the operation unit 1221. For example, when an operation instructing the blood pressure measurement is performed, the instruction input unit 1610 provides the blood-pressure-measuring control unit 1608 with a start instruction of the blood pressure measurement. The instruction input unit 1610 and the operation unit 1221 correspond to operation input means according to the present invention.

The blood-pressure-measuring control unit 1608 controls the pump drive circuit 1406 to perform the blood pressure measurement. When the blood-pressure-measuring control unit 1608 receives the start instruction of the blood pressure measurement from the instruction input unit 1610, the blood-pressure-measuring control unit 1608 drives the pump 1403 via the pump drive circuit 1406. Accordingly, supply of air to the pressing cuff 1401 starts. The pressing cuff 1401 is inflated, whereby the upper arm of the user is compressed. The blood-pressure-measuring control unit 1608 monitors the cuff pressure using the pressure sensor 1402. The blood-pressure-measuring control unit 1608 calculates the blood pressure value using the oscillometric method based on a pressure signal output from the pressure sensor 1402 in pressurizing processing of supplying air to the pressing cuff 1401. The blood pressure value includes systolic blood pressure (SBP) and diastolic blood pressure (DBP), but is not limited to these. The blood-pressure-measuring control unit 1608 causes the actually measured blood pressure value storage unit 1609 to store the calculated blood pressure value in association with time information. The blood-pressure-measuring control unit 1608 can calculate a pulse rate while simultaneously with the blood pressure value. The blood-pressure-measuring control unit 1608 stops the pump 1403 via the pump drive circuit 1406 when calculation of the blood pressure value is completed. Thus, air is exhausted from the pressing cuff 1401 through the valve 1404.

The display control unit 1607 controls the display unit 1222. For example, the display control unit 1607 receives an instruction signal from the instruction unit 160 and causes the display unit 1222 to display a message included in the instruction signal. For example, the display control unit 1607 displays the blood pressure measurement result on the display unit 1222 after the blood pressure measurement by the blood-pressure-measuring control unit 1608 has been completed.

The calibration processing unit 170 calibrates the blood pressure calculation formula based on the estimated blood pressure value obtained by the blood pressure value calculation unit 134 and the actually measured blood pressure value obtained by the blood-pressure-measuring control unit 1608. Besides, the blood pressure calculation formula may be calibrated by the calibration processing unit 170, for example, so as to be performed as an initial setting when the user attaches the blood-pressure-measuring device 10. The correlation between the pulse transit time and blood pressure values varies from individual to individual. Additionally, the correlation relationship changes according to the state in which the blood-pressure-measuring device 10 is attached to the upper arm of the user. For example, even within an identical user, the correlation varies between positioning of the blood-pressure-measuring device 10 closer to the shoulder and positioning of the blood-pressure-measuring device 10 closer to the elbow. To reflect such a variation in correlation, the blood pressure calculation formula is calibrated.

The calibration processing unit 170 also changes the reference value stored in the calibration determination reference value storage unit 1611. Specifically, for example, the difference between the estimated blood pressure value when the calibration determination unit 150 determines to acquire the actually measured blood pressure value and the actually measured blood pressure value is calculated, and when the difference is large, the reference value is changed so as to increase a frequency of calibration, and when the difference is small, the reference value is changed so as to decrease the frequency of calibration.

Also, the present embodiment describes an example in which all the functions of the blood-pressure-measuring device 10 are realized by a general-purpose processor. However, a portion or all of the functions may be realized by one or more dedicated processors.

### Operation Example

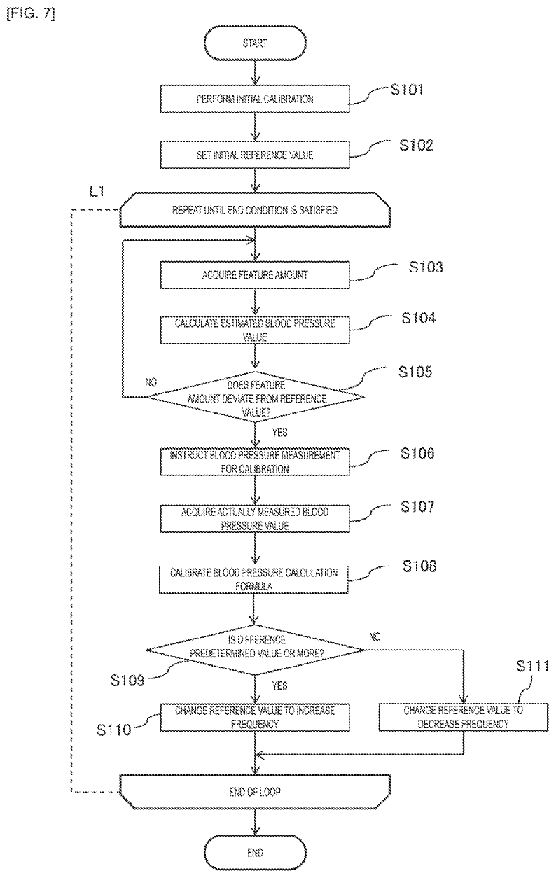

Next, the operation example of the blood-pressure-measuring device 10 according to the present embodiment will be described with reference to FIG. 7. FIG. 7 is a flowchart depicting an example of a flow of processing performed by the blood-pressure-measuring device 10. First, when the user uses the blood-pressure-measuring device 10 first, initial calibration of the blood pressure calculation formula is performed (S101). In this processing, the control unit 1501 operates as the calibration processing unit 170. Assuming that N is the number of the parameters included in the blood pressure calculation formula, N or more sets of the measurement value of the pulse transit time and the measurement value for the blood pressure are required. The blood pressure calculation Formula (1) described above includes the two parameters A₁ and A₂. In this case, for example, the control unit 1501 acquires the set of that measurement value of the pulse transit time and the measurement value of the blood pressure while the user is at rest, subsequently the user is caused to exercise, and the control unit 1501 acquires the set of the measurement value of the pulse transit time and the measurement value of the blood pressure after the exercise. Thus, the two sets of the measurement value of the pulse transit time and the measurement value of the blood pressure are acquired. The control unit 1501 determines the parameters A₁ and A₂ based on the acquired two sets of the measurement value of the pulse transit time and the measurement value of the blood pressure.

Subsequently, further, a reference value for determining the necessity to acquire the actually measured blood pressure value is set (S102). The reference value at this time may be calculated according to the determined parameters A₁, A₂, or a general-purpose reference value may be set in advance. The reference value set here is stored in the calibration determination reference value storage unit 1611.

After the initial calibration is completed, the blood pressure measurement (estimation) based on the pulse transit time can be performed, and the following loop processing L1 is repeated until a predetermined end condition is satisfied, whereby the continuous, non-invasive blood pressure measurement is performed.

In the loop processing L1, the following processing is repeatedly performed. First, the control unit 1501 continuously calculates the pulse transit time for calculating the estimated blood pressure value (S103). Further, the estimated blood pressure value is calculated based on the calculated pulse transit time and the blood pressure calculation formula stored in the blood pressure calculation formula storage unit (S104). Then, processing that determines whether or not the next calculated pulse transit time deviates from the reference value stored in the calibration determination reference value storage unit 1611 is performed (S105). The reference value may be, for example, an upper limit threshold or a lower limit threshold of the pulse transit time. Alternatively, the reference value may be upper and lower limit thresholds defining a predetermined value range. That is, in step S105, it is determined whether or not the pulse transit time exceeds the reference value when the reference value is the upper limit value, whether or not the pulse transit time is less than the reference value when the reference value is the lower limit value, and whether or not the pulse transit time falls within the predetermined value range between the upper and lower limit thresholds when the reference values are the upper and lower limit thresholds.

When it is determined in step S105 that the reference value is not deviated, the flow returns to the processing of step S103 and the subsequent processing is repeated. On the other hand, when it is determined in step S105 that the reference value is deviated, the processing proceeds to step S106, and it is determined that the actually measured blood pressure value is to be acquired by the second blood-pressure-measuring unit 140 for calibration of the blood pressure calculation formula. In the processing of step S105, the control unit 1501 functions as the calibration determination unit 150.

In step S106, the control unit 1501 performs control for outputting information instructing the blood pressure measurement by the second blood-pressure-measuring unit 140. At step S106, the control unit 1501 operates as the instruction unit 160. When the start instruction of the blood pressure measurement by the user is received via the operation unit 1221, the second blood-pressure-measuring unit 140 performs processing of acquiring the actually measured blood pressure value (S107). In step S107, the control unit 1501 operates as the blood-pressure-measuring control unit 1608.

When the actually measured blood pressure value is acquired, the control unit 1501 calibrates the blood pressure calculation formula stored in the blood pressure calculation formula storage unit 1605 based on the actually measured blood pressure value (S108), and performs processing for determining whether or not the difference between the estimated blood pressure value and the actually measured blood pressure value is equal to or more than a predetermined threshold (S109).

Here, when the difference is equal to or more than the threshold, the reference value stored in the calibration determination reference value storage unit 1611 is changed such that the frequency at which the instruction unit 160 instructs acquisition of the actually measured blood pressure value increases (S110). Specifically, when the reference values are the upper and lower limit thresholds of the pulse transit time, the reference values are changed such that the upper threshold is decreased and the lower threshold is increased, that is, the value range defined by the upper and lower limit thresholds is decreased. In this way, the calculated pulse transit time is more likely to deviate from the upper and lower limit thresholds than before the reference value is changed, and as a result, the frequency at which the instruction unit 160 instructs acquisition of the actually measured blood pressure value increases.

On the other hand, when it is determined in step S108 that the difference is less than the threshold, the reference value is changed such that the frequency at which the instruction unit 160 instructs acquisition of the actually measured blood pressure value decreases (S111). To be specific, when the reference values are the upper and lower limit thresholds of the pulse transit time contrary to the case of step S110, the reference values are changed such that the upper threshold is increased and the lower threshold is decreased, that is, the value range defined by the upper and lower limit thresholds is expanded. In this way, the calculated pulse transit time is less likely to deviate from the upper and lower limit thresholds than before the reference value is changed, and as a result, the frequency at which the instruction unit 160 instructs acquisition of the actually measured blood pressure value decreases.

When the processing of Step S110 or Step S111 is performed, a series of the loop processing L1 is ended, and the processing returns to the start end of the loop processing L1 (that is, Step S103) again to perform the new loop processing L1. In the processing from step S108 to step S111, the control unit 1501 functions as the calibration processing unit 170.

Note that the processing procedure depicted in FIG. 7 is an example, and the processing order or the content of each processing can be changed as appropriate. For example, in the processing procedure, after the blood pressure calculation formula is calibrated in step S108, the processing proceeds to step S109 to determine whether or not the difference between the actually measured blood pressure value and the estimated blood pressure value is equal to or more than the predetermined threshold. However, the order may be switched and the processing of determine whether or not the difference between the actually measured blood pressure value and the estimated blood pressure value is equal to or more than the predetermined threshold may be performed first. When the difference is then less than the predetermined threshold, the blood pressure calculation formula can be avoided to be calibrated.

In addition, in the above-described processing procedure, when the difference is equal to or more than the predetermined threshold in step S109, the reference value is changed such that the frequency of calibration increases, otherwise the reference value is changed such that the frequency of calibration decreases, but the thresholds may be provided for the upper limit and the lower limit. That is, the reference value may be changed so as to increase the frequency of calibration when the difference is equal to or more than the upper limit threshold, the reference value may be changed so as to decrease the frequency of calibration when the difference is the lower limit threshold or less, and the reference value may be not changed when the difference does not deviate from the upper and lower limit thresholds.

### Effect

As described above, in the blood-pressure-measuring device 10 according to the present embodiment, both the electrode group 1311 and the sensor unit 1322 of the pulse wave sensor 1321 are provided on the belt 121. Thus, both the electrode group 1311 and the pulse wave sensor 1321 are mounted on the user simply by winding the belt 121 around the upper arm. Thus, the blood-pressure-measuring device 10 can be easily attached to the user. Since the user only needs to attach one device, a feeling of rejection of the user for the attachment of the blood-pressure-measuring device 10 is reduced.

Since the blood-pressure-measuring device 10 is attached to the upper arm, the blood pressure measurement is performed at substantially the same height as the heart. Accordingly, it is not necessary to perform height correction on the acquired blood pressure measurement result. In addition, when the blood-pressure-measuring device 10 is an upper arm type, the blood-pressure-measuring device 10 can be hidden by a sleeve of clothes, and it is possible to make the attachment of the blood-pressure-measuring device 10 inconspicuous.

Further, since the pulse transit time is calculated based on the electrocardiogram and the pulse wave signal obtained for the upper arm, the pulse transit time can be obtained for a long distance from the heart to the upper arm. This improves robustness against an error that occurs when the time difference between the waveform feature point in the electrocardiogram and the waveform feature point in the pulse wave signal is calculated. Further, the electrode group 1311 is disposed on the central side portion 1217A of the belt 121, and the sensor unit 1322 of the pulse wave sensor 1321 is disposed on the peripheral side portion 1217B of the belt 121. In this arrangement, the further long pulse transit distance is ensured and the electrocardiogram with the high SN ratio is acquired. Thus, the robustness is further improved. This allows accurately measuring the pulse transit time and improves the reliability of the blood pressure value calculated based on the pulse transit time.

In the present embodiment, since the blood pressure measurement based on the pulse transit time and the blood pressure measurement using the oscillometric method can be performed by one device, a convenience for the user is high. The second blood-pressure-measuring unit 140 is integrated with the first blood-pressure-measuring unit 130, and the blood pressure calculation formula is calibrated based on the actually measured blood pressure value obtained by the second blood-pressure-measuring unit 140. Therefore, the blood-pressure-measuring device 10 alone can calibrate the blood pressure calculation formula. For this reason, the blood pressure calculation formula can be calibrated easily.

Then, based on the result of the continuous blood pressure measurement by the first blood-pressure-measuring unit 130, it is determined whether or not the actually measured blood pressure value of the user should be acquired (that is, whether or not it is necessary to calibrate the algorithm for calculating the blood pressure value), and when the condition is satisfied, the user is notified that the blood pressure measurement by the second blood-pressure-measuring unit 140 should be performed. Therefore, it is possible to cause the user to perform the accurate blood pressure measurement under a situation in which the blood pressure measurement is recommended.

Further, since the reference value of the predetermined feature amount serving as a criterion for determining whether or not the actually measured blood pressure value should be acquired changes in accordance with the difference value between the actually measured blood pressure value and the estimated blood pressure value (that is, the accuracy of the estimated blood pressure value), the frequency of acquiring the actually measured blood pressure value can be optimized. Accordingly, it is possible to provide a technology that allows improving the accuracy of blood pressure estimation according to the user by repeating the calibration of the blood pressure value calculation algorithm, and optimizing the frequency of calibrating the blood pressure value calculation algorithm.

### Modified Example

In the embodiment described above, the pulse wave sensor employs an impedance method in which a change in impedance resulting from a change in volume of the artery is detected. Also, the pulse wave sensor may adopt another measurement method such as a photoelectric method, a piezoelectric method, or a radio wave method. In an embodiment employing the photoelectric method, the pulse wave sensor includes: a light emitting element that radiates light toward the artery passing through a target measurement site, and a photodetector for detecting reflected light or transmitted light of the light, and the pulse wave sensor detects a change in light intensity resulting from a change in volume of the artery. In an embodiment employing the piezoelectric method, the pulse wave sensor includes a piezoelectric element provided on the belt to be in contact with the target measurement site and detects a change in pressure resulting from a change in volume of the artery. In an embodiment employing a radio wave method, the pulse wave sensor includes: a transmission element that transmits a radio wave toward the artery passing through a target measurement site and a receiving element that receives a reflection wave of the radio wave, and the pulse wave sensor detects a phase shift between the transmission wave and the reflection wave associated with the change in volume of the artery.

The blood-pressure-measuring device 10 may further include a pressing cuff for adjusting the contact state between the sensor unit 1322 of the pulse wave sensor 1321 and the upper arm, a pump that supplies air to the pressing cuff, a pump drive circuit that drives the pump, and a pressure sensor for detecting the pressure in the pressing cuff. This pressing cuff is provided at the peripheral side end portion 1218C of the belt 121. In this case, the pressing cuff 1401 is provided at the intermediate portion 1218B of the belt 121, for example.

A portion of the blood-pressure-measuring device involved in the measurement of the pulse transit time may be implemented as a single device. In an embodiment, a pulse transit time measurement device including the belt unit 120, the electrocardiogram acquisition unit 131, the pulse wave signal acquisition unit 132, and the pulse transit time calculation unit 133 is provided. The pulse transit time measurement device may further include the calibration determination unit 150 and the instruction unit 160. The pulse transit time measurement device may further include a pressure cuff to press the electrodes 1312 and the pulse wave sensor 1321 against the upper arm, a pump, and a pump drive circuit.

The blood-pressure-measuring device 10 need not include the second blood-pressure-measuring unit 140. In an embodiment in which the blood-pressure-measuring device 10 does not include the second blood-pressure-measuring unit 140, a blood pressure value obtained by measurement with another blood pressure monitor needs to be input to the blood-pressure-measuring device 10 for calibration of the blood pressure calculation formula.

### Second Embodiment

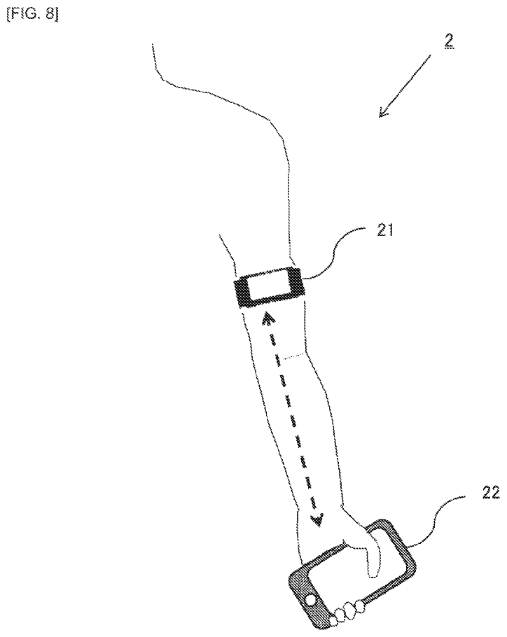

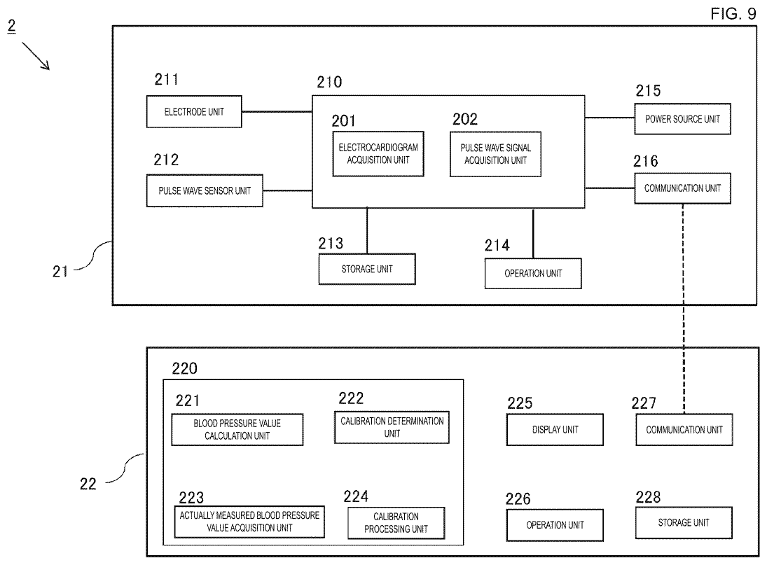

In the first embodiment, the present invention is applied as the blood-pressure-measuring device, and all of the functions, including the storage unit, the blood pressure value calculation unit, the display unit, and the like are integrated into one device. However, the present invention can be applied as a blood-pressure-measuring system in which a part of such configurations and functions are separated. FIG. 8 and FIG. 9 illustrate examples of such a blood-pressure-measuring system.

FIG. 8 illustrates an outline of a blood-pressure-measuring system 2 according to the present embodiment. As illustrated in FIG. 8, the blood-pressure-measuring system 2 includes a sensor device 21 attached to the upper arm of the user and an information processing terminal 22 that processes biological information acquired by the sensor device 21. Although not illustrated, the sensor device 21 is a wearable device including a plurality of electrodes (electrocardiographic sensors) and a pulse wave sensor, and is used while being fixed to the upper arm of the user with fixing means, such as a belt. The information processing terminal 22 may be of any type as long as it can communicate with the sensor device 21. For example, as illustrated in FIG. 8, a smartphone can be used as the information processing terminal 22.

FIG. 9 is a block diagram illustrating functional configurations of the sensor device 21 and the information processing terminal 22 of the blood-pressure-measuring system 2. The sensor device 21 includes functional units of an electrode unit 211, a pulse wave sensor unit 212, a control unit 210, a storage unit 213, an operation unit 214, a power source unit 215, and a communication unit 216. Further, the control unit 210 includes, as its functional modules, an electrocardiogram acquisition unit 201 and a pulse wave signal acquisition unit 202.

For example, a photoelectric method can be employed for the pulse wave sensor unit 212 and the pulse wave signal acquisition unit 202 in the sensor device 21. Specifically, there are provided a light emitting element that irradiates light toward the artery passing through the target measurement site and a photodetector for detecting reflected light or transmitted light of the light, and a change in light intensity resulting from a change in the arterial volume is detected (all of them are not illustrated). In addition, since the electrode unit 211 and the electrocardiogram acquisition unit 201 can have the same configuration as that of the blood-pressure-measuring device 10 of the first embodiment, a detailed description thereof will be omitted.

Furthermore, the storage unit 213 includes only a main storage device, such as a RAM or a ROM, and has a limited storage capacity. The operation unit 214 also has a limited configuration, such as a power switch, and has a simple configuration. The power source unit 215 can be, for example, a rechargeable secondary battery. The communication unit 216 includes a wired communication module and/or a wireless communication module. Note that a connection terminal for wired communications may also serve as a charging terminal of the power source unit 215.

As described above, the sensor device 21 in the present embodiment is configured to have only a very limited function for acquiring the biological information for calculating the estimated blood pressure value. Therefore, an electrocardiographic signal and the pulse wave signal measured by each sensor unit is transmitted to the information processing terminal 22 via the communication unit 216 in real time.

The information processing terminal 22 includes respective functional units of a control unit 220, a display unit 225, an operation unit 226, a communication unit 227, and a storage unit 228. The control unit 220 includes functional modules of a blood pressure value calculation unit 221, a calibration determination unit 222, an actually measured blood pressure value acquisition unit 223, and a calibration processing unit 224.

The information processing terminal 22 communicates with the sensor device 21 via the communication unit 227 and receives the electrocardiographic signal and the pulse wave signal of the user measured by the sensor device 21. The communication standard is not particularly limited, but communication can be performed by a wireless communication standard such as Bluetooth (registered trademark), Wi-Fi (registered trademark), or infrared communication. Note that a hardware configuration of the information processing terminal 22 is the same as the configuration of a smartphone. For example, a touch panel display serves as both the display unit 225 and the operation unit 226.

The biological information received from the sensor device 21 via the communication unit 227 is stored in the storage unit 228, and each processing, such as calculation of the estimated blood pressure value, is performed based on the stored information. Similar to the storage unit 1505 of the blood-pressure-measuring device 10 according to the first embodiment, the storage unit 228 stores not only the electrocardiogram and the pulse wave signal but also information, such as the algorithm for calculating the blood pressure value, the determination reference value for determining whether or not the calibration needs to be performed, the estimated blood pressure value, and the actually measured blood pressure value.

The respective blood pressure value calculation unit 221, calibration determination unit 222, and calibration processing unit 224 are functional modules that perform the calculation processing of the estimated blood pressure value, the determination processing of whether or not the algorithm calibration needs to be performed using the actually measured blood pressure value, the algorithm calibration processing for blood pressure calculation, and reference value change processing for determining the necessity of the calibration, similarly to the blood-pressure-measuring device 10 of the first embodiment. Since the processing is the same as those in the first embodiment, a repeated description is omitted here.

When the calibration determination unit 222 determines that the blood pressure calculation algorithm calibration using the actually measured blood pressure value is necessary, the actually measured blood pressure value acquisition unit 223 performs the processing of acquiring the actually measured blood pressure value. In the present embodiment, for example, the display unit 225 or a speaker (not illustrated) notifies the user that the actually measured blood pressure value should be input. The user measures the actually measured blood pressure value using another device (not illustrated) that allows accurate blood pressure measurement, such as an oscillometric method, and inputs the blood pressure value to the information processing terminal 22 by operating the operation unit 226. That is, the actually measured blood pressure value acquisition unit 223 acquires the actually measured blood pressure value via the operation unit 226. The acquired actually measured blood pressure value is stored in the storage unit 228.

The blood-pressure-measuring system 2 of the present embodiment has a configuration in which sensing of the biological information (for example, the electrocardiogram and the pulse wave signal) for continuous calculation of the estimated blood pressure value is performed by the sensor device 21, and actual blood pressure value calculation processing, calibration necessity determination processing, algorithm calibration processing, and the like are performed by the information processing terminal 22. According to such a configuration, the configuration of the wearable device can be simplified, and a burden on the user related to the attachment of the device can be further reduced. In addition, since an already existing information processing terminal, such as a smartphone, can be utilized, costs when the user introduces the system can be reduced.

### Third Embodiment

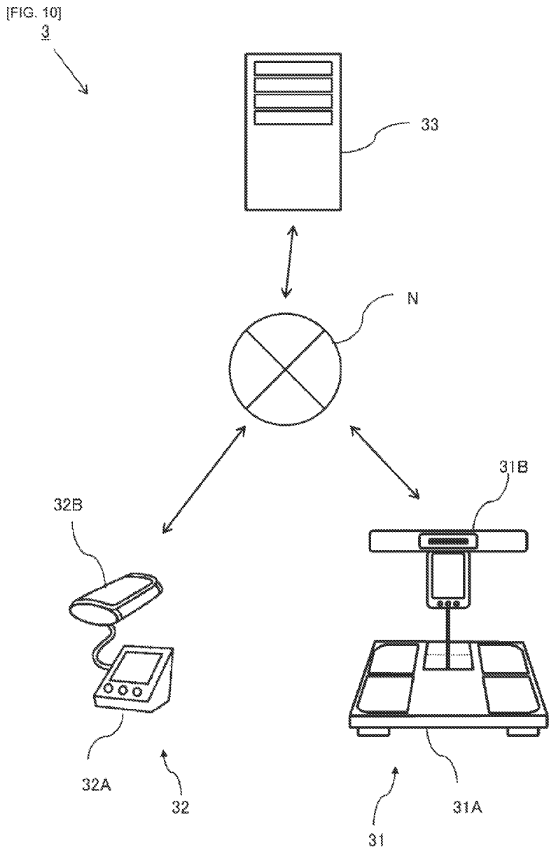

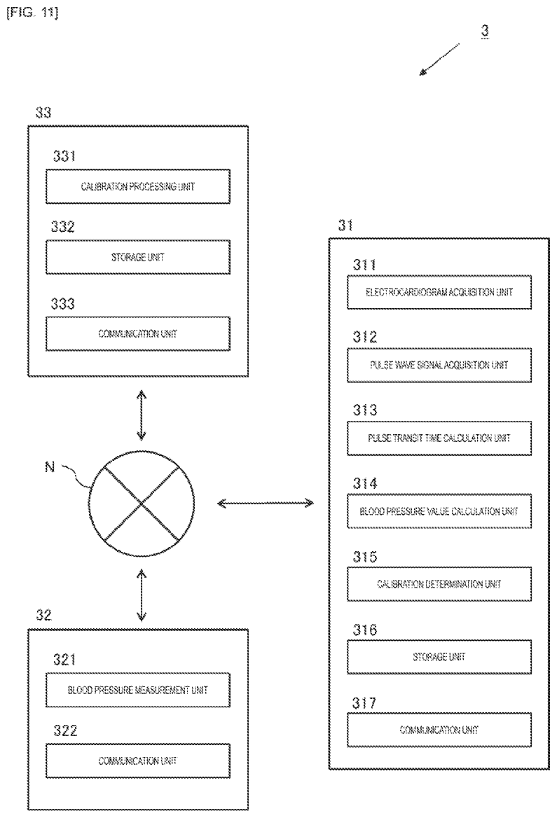

In the above-described embodiment, an example in which the blood pressure is measured continuously on a daily basis using the wearable dedicated blood-pressure-measuring device has been described, but the present invention can be performed without using the device dedicated to blood pressure measurement. FIG. 10 and FIG. 11 illustrate examples of such a blood-pressure-measuring system.

FIG. 10 illustrates an outline of a blood-pressure-measuring system 3 according to the present embodiment. As illustrated in FIG. 10, the blood-pressure-measuring system 3 has a configuration in which a body composition meter 31, a blood-pressure-measuring device 32, and a server 33 are connected via a network N. As the network N, for example, any communication network including a wide area network (WAN) that is a worldwide public communication network, such as the Internet, may be employed. In addition, the network N may include a telephone communication network, such as a mobile phone, and a wireless communication network, such as Wi-Fi (registered trademark).

The body composition meter 31 generally includes a main body portion 31A and a handle portion 31B. Although not illustrated, in addition to communication means for communication, sensors (for example, strain gauges, electrodes, velocity sensors, and the like) for acquiring various pieces of biological information, such as a body weight, a body fat percentage, an electrocardiogram, a pulse wave signal, and ballistocardiogram (BCG), an output unit, such as a liquid crystal display, an input unit, such as an operation button, a power supply unit, and the like are provided.

The blood-pressure-measuring device 32 is a general home blood-pressure-measuring device schematically including the main body portion 32A and a cuff portion 32B, and includes respective elements for measuring the blood pressure by an oscillometric method, such as a pressure sensor, a pressing cuff, and a pump, an output unit, such as a liquid crystal display, and an input unit, such as an operation button.

The server 33 is configured by a general server computer, and includes a processor, such as a CPU, a main storage device, such as a RAM and a ROM, an auxiliary storage device, such as an EPROM, an HDD, and a removable medium, and the like.

FIG. 11 is a block diagram illustrating a functional configuration of the blood-pressure-measuring system 3. The body composition meter includes an electrocardiogram acquisition unit 311, a pulse wave signal acquisition unit 312, a pulse transit time calculation unit 313, a blood pressure value calculation unit 314, a calibration determination unit 315, a storage unit 316, and a communication unit 317.

The electrocardiogram acquisition unit 311 acquires the electrocardiogram of the user via electrodes disposed on an upper surface of the main body portion 31A and the handle portion 31B of the body composition meter 31. The pulse wave signal acquisition unit 312 acquires pulse wave signals (peripheral pulse waves) of the user via a pulse wave sensor disposed on the handle portion 31B. The pulse wave sensor may be an impedance type sensor or a photoelectric type sensor. The acquired electrocardiogram and pulse wave signal are stored in the storage unit 316. Similarly to the blood-pressure-measuring device 10 of the first embodiment, the storage unit 316 stores the blood pressure calculation algorithm, the determination reference value for determining whether or not the calibration needs to be performed, and the like, in addition to the biological information.

The pulse transit time calculation unit 313 reads the electrocardiogram and the pulse wave signal from the storage unit 316 and calculates the pulse transit time based on the time difference between the waveform feature point in the electrocardiogram and the waveform feature point in the pulse wave signal. The blood pressure value calculation unit 314 calculates the blood pressure value based on the calculated pulse transit time and the blood pressure calculation algorithm stored in the storage unit 316. The calibration determination unit 315 determines whether or not the blood pressure calculation algorithm should be calibrated based on the calculated pulse transit time and the predetermined reference value stored in the storage unit 316. Since each of the processing is the same as those in the case of the blood-pressure-measuring device 10 of the first embodiment, a detailed description thereof will be omitted here.

When the calibration determination unit 315 determines that the blood pressure calculation algorithm needs to be calibrated, the calibration determination unit 315 notifies the user of the fact via a display unit (not illustrated) or the like, and transmits the estimated blood pressure value when the calibration determination unit 315 determines that the calibration is necessary to the server 33 via the communication unit 317 and the network N.

The blood-pressure-measuring device 32 includes a blood-pressure-measuring unit 321 and a communication unit 322 as functional units. The blood-pressure-measuring unit 321 is a functional unit that performs accurate blood pressure measurement by means, such as an oscillometric method, and can have a configuration similar to that of the second blood-pressure-measuring unit 140 in the blood-pressure-measuring device 10 of the first embodiment, and thus description thereof is omitted here. The actually measured blood pressure value measured by the blood-pressure-measuring unit 321 is transmitted to the server 33 via the communication unit 322 and the network N.

The server 33 includes respective functional units of a calibration processing unit 331, a storage unit 332, and a communication unit 333. The information (the estimated blood pressure value, the actually measured blood pressure value, and the like) transmitted from the body composition meter 31 and the blood-pressure-measuring device 32 and received by the communication unit 333 is stored in the storage unit 332. The calibration processing unit 331 performs processing for calibrating the blood pressure calculation algorithm of the body composition meter 31 based on the estimated blood pressure value and the actually measured blood pressure value stored in the storage unit 332. Specifically, a more appropriate parameter value is calculated based on the estimated blood pressure value and the actually measured blood pressure value, and data of the new parameter thus calculated is transmitted to the body composition meter 31 via the communication unit 333 and the network N. Then, the blood pressure calculation algorithm stored in the storage unit 316 of the body composition meter 31 is updated to a new algorithm using the new parameters, whereby the blood pressure calculation algorithm is calibrated.

Similarly to the first and second embodiments, the calibration processing unit 331 also performs change processing of the reference value used in the determination processing performed by the calibration determination unit 315. In this case as well, as in the case of the calibration of the algorithm, the server 33 calculates a new reference value, transmits the calculated new reference value to the body composition meter 31, and stores the new reference value in the storage unit 316, thereby changing the reference value.

As described above, in the blood-pressure-measuring system 3 according to the present embodiment, the biological information for calculating the estimated blood pressure value is acquired using the general-purpose body composition meter 31 instead of a dedicated device. Further, the function of the calibration processing unit 331 is not provided in the body composition meter 31 but is performed in the server 33. This eliminates the need for performing complicated arithmetic processing for calibration of the algorithm on the measurement instrument side, and thus it is possible to measure (estimate) the blood pressure value using a general-purpose body composition meter, and to calibrate the algorithm as appropriate. That is, even when a general-purpose body composition meter is used, the accuracy of the estimated blood pressure value can be kept high. In the present embodiment, the body composition meter 31 includes the handle portion 31B, but it is also possible to use a body composition meter that does not include the handle portion 31B.

### Other Points

The description of the embodiments described above is merely illustrative of the present invention, and the present invention is not limited to the specific embodiments described above. Within the scope of the technical idea of the present invention, various modifications and combinations may be made. For example, in each of the examples, the feature amount used to determine the necessity of the calibration of the blood pressure calculation algorithm is the pulse transit time. However, the necessity of the calibration of the algorithm may be determined based on a feature amount other than that. For example, in the third embodiment, information, such as a weight, a BMI, a ballistocardiogram, and a pulse wave velocity (PWV), can be acquired from various sensors included in the body composition meter 31, and these feature amounts can also be used for blood pressure estimation. That is, these feature amounts can also be used to determine the necessity of the calibration of the algorithm. In addition, a height at an inflection point of a pulse wave or an electrocardiographic waveform, a gradient between two inflection points, an area between the two inflection points, a ratio thereof, or the like may be used as a feature amount for determining the necessity of the calibration of the algorithm. In addition, information on a heart rate (such as a difference from a previous beat and a difference from an average value of beats), attribute information of an individual user (such as a height, an age, and medication history), information on a situation at the time of measurement (such as an amount of activity of the user and a posture), environment information (such as season and an external temperature), or the like may be used as the feature amount.

In addition, in the first embodiment, in the case where the reference value is the upper and lower limit thresholds for the pulse transit time, the example in which both the upper and lower limit thresholds are changed has been described. However, the change of the reference value is not limited thereto, and various patterns can be set. For example, the reference value can be set as only the upper limit threshold or only the lower limit threshold. When the lower limit threshold is set, the frequency of calibration can be increased by increasing the reference value, and the frequency of calibration can be decreased by decreasing the reference value. When the reference value is the upper and lower limit thresholds, only the upper limit threshold or only the lower limit threshold may be changed. Even in such a case, it is possible to change the width of the value range in which the feature amount falls within, and it is possible to change the frequency of calibration in accordance therewith.

In addition, the device that measures the biological information is not limited to those exemplified in the above-described respective embodiments, and may be a device, such as a so-called smart watch. The measurement target site is not limited to an upper arm or a wrist and may be a measurement instrument attached to another site, such as thigh or ankle, may be used.

### REFERENCE NUMERALS LIST

10 Blood-pressure-measuring device, 120 Belt unit, 121 Belt, 122 Body, 130 First blood-pressure-measuring unit, 131 Electrocardiogram acquisition unit, 132 Pulse wave signal acquisition unit, 133 Pulse transit time calculation unit, 134 Blood pressure value calculation unit, 140 Second blood-pressure-measuring unit, 150 Calibration determination unit, 160 Instruction unit, 1210A Inner cloth, 1210B Outer cloth, 1213 Loop surface, 1214 Hook surface, 1221 Operation unit, 1222 Display unit, 1311 Electrode group, 1312 Electrode, 1313 Switch circuit, 1314 Subtraction circuit, 1315 Analog front end, 1321 Pulse wave sensor, 1322 Sensor unit, 1323A to 1323D Electrode, 1324 Energization and voltage detection circuit, 1401 Pressing cuff, 1402 Pressure sensor, 1403 Pump, 1404 Valve, 1405 Oscillation circuit, 1406 Pump drive circuit, 1501 Control unit, 1502 CPU, 1503 RAM, 1504 ROM, 1505 Storage unit, 1506 Battery, 1507 Communication unit, 1601 Electrocardiogram measurement control unit, 1602 Electrocardiogram storage unit, 1603 Pulse wave measurement control unit, 1604 Pulse wave signal storage unit, 1605 Blood pressure calculation formula storage unit, 1606 Estimated blood pressure value storage unit, 1607 Display control unit, 1608 Blood-pressure-measuring control unit, 1609 Actually measured blood pressure value storage unit, 1610 Instruction input unit, 1611 Calibration determination reference value storage unit
UA Upper arm, UAA Brachial artery, UAB Humerus
2, 3 Blood-pressure-measuring system
21 Sensor device, 22 Information processing terminal
31 Body composition meter, 31A Main body portion, 31B handle portion
32 Blood-pressure-measuring device, 32A Main body portion, 32B Cuff portion
33 Server
N Network

1. A blood-pressure-measuring device, comprising:
a feature amount acquisition unit that acquires one or more feature amounts related to estimation of a blood pressure value of a human body;
a blood pressure value calculation unit that calculates an estimated blood pressure value based on the feature amount;
an actually measured blood pressure value acquisition unit that acquires an actually measured blood pressure value measured by a method different from the calculation by the blood pressure value calculation unit;
a calibration determination unit that determines whether or not the feature amount acquired by the feature amount acquisition unit deviates from a predetermined reference value, the calibration determination unit determining to acquire the actually measured blood pressure value when the calibration determination unit has determined that the feature amount deviates; and
a calibration processing unit that calibrates a calculation algorithm of the estimated blood pressure value by the blood pressure value calculation unit using the actually measured blood pressure value, wherein
the calibration processing unit changes the reference value based on the actually measured blood pressure value acquired by the determination by the calibration determination unit and the estimated blood pressure value calculated using the feature amount deviated from the reference value.

2. The blood-pressure-measuring device according to claim 1, wherein
when a difference between the actually measured blood pressure value acquired by the determination by the calibration determination unit and the estimated blood pressure value calculated using the feature amount deviating from the reference value is a predetermined threshold or less, the calibration processing unit changes the reference value to a value that decreases a frequency determined when the actually measured blood pressure value is acquired.

3. The blood-pressure-measuring device according to claim 2, wherein
the calibration processing unit;
increases the value of the reference value when the reference value is an upper limit threshold for the feature amount;
decreases the value of the reference value when the reference value is a lower limit threshold for the feature amount; and
increases the upper limit threshold and/or decreases the lower limit threshold when the reference values are upper and lower limit thresholds that define a value range for the feature amount.

4. The blood-pressure-measuring device according to claim 1, wherein
when a difference between the actually measured blood pressure value acquired by the determination by the calibration determination unit and the estimated blood pressure value calculated using the feature amount deviating from the reference value exceeds a predetermined threshold, the calibration processing unit changes the reference value to a value that increases a frequency determined when the actually measured blood pressure value is acquired.

5. The blood-pressure-measuring device according to claim 4, wherein
the calibration processing unit;
decreases the value of the reference value when the reference value is an upper limit threshold for the feature amount;
increases the value of the reference value when the reference value is a lower limit threshold for the feature amount; and
decreases the upper limit threshold and/or increases the lower limit threshold when the reference values are upper and lower limit thresholds that define a value range for the feature amount.

6. The blood-pressure-measuring device according to claim 1, further comprising output means, wherein
when the calibration determination unit has determined to acquire the actually measured blood pressure value, information indicating that the actually measured blood pressure value is to be acquired is output from the output means.

7. The blood-pressure-measuring device according to claim 1, further comprising blood-pressure-measuring means that measures the actually measured blood pressure value, wherein
when the calibration determination unit has determined to acquire the actually measured blood pressure value, the actually measured blood pressure value acquisition unit acquires the actually measured blood pressure value by measuring the actually measured blood pressure value by the blood-pressure-measuring means.

8. The blood-pressure-measuring device according to claim 1, further comprising:
blood-pressure-measuring means for measuring the actually measured blood pressure value; and
operation input means, wherein
the actually measured blood pressure value acquisition unit acquires the actually measured blood pressure value by measuring the actually measured blood pressure value by the blood-pressure-measuring means when an input instructing the measurement of the actually measured blood pressure value is received via the operation input means.

9. A blood-pressure-measuring system, comprising:
feature amount acquisition means that acquires one or more feature amounts related to estimation of a blood pressure value of a human body;
blood pressure value calculation means that calculates an estimated blood pressure value based on the feature amount;
actually measured blood pressure value acquisition means that acquires an actually measured blood pressure value measured by a method different from the calculation by the blood pressure value calculation means;
calibration determination means that determines whether or not the feature amount acquired by the feature amount acquisition means deviates from a predetermined reference value, the calibration determination means determining to acquire the actually measured blood pressure value when the calibration determination means has determined that the feature amount deviates; and
calibration processing means that calibrates a calculation algorithm of the estimated blood pressure value by the blood pressure value calculation means using the actually measured blood pressure value, wherein
the calibration processing means changes the reference value based on the actually measured blood pressure value acquired by the determination by the calibration determination means and the estimated blood pressure value calculated using the feature amount deviated from the reference value.

10. The blood-pressure-measuring system according to claim 9, comprising:
a measurement instrument that includes one or more sensors that detect at least the feature amount; and
an information processing device that includes at least the calibration processing means.

11. The blood-pressure-measuring system according to claim 10, wherein
the measurement instrument further includes blood-pressure-measuring means for measuring the actually measured blood pressure value.

12. The blood-pressure-measuring system according to claim 10, wherein
the measurement instrument is a wearable device constantly attachable to a human body.
